(19) United States
(12) Patent Application Publication
(10) Pub. No.: US 2011/0186512 A1
(43) Pub. Date: Aug. 4, 2011

(54) PROCESS FOR DISINFECTING A FILTRATION WORKS FOR PRETREATMENT OF SALTWATER, AND INSTALLATION FOR THE IMPLEMENTATION THEREOF

(75) Inventor: Véronique Bonnelye, F-Conflans Sainte Honorine (FR)

(73) Assignee: DEGREMONT, Rueil-Malmaison (FR)

(21) Appl. No.: 13/001,171

(22) PCT Filed: Jun. 24, 2009
(86) PCT No.: PCT/IB2009/052704

§ 371 Date: Mar. 8, 2011

(30) Foreign Application Priority Data
Jun. 26, 2008 (FR) 0803591

(51) Int. Cl. B01D 65/02; C02F 1/44; B01D 24/46; A61L 2/18

(52) U.S. Cl. 210/636; 210/258

(57) ABSTRACT

Method for disinfecting a filtration works (F) for the pretreatment of salt water, particularly seawater, upstream of a water desalination unit (1) using a reverse osmosis membrane, whereby an operation of disinfecting the filtration works is carried out periodically by adding a bactericidal agent to the water that is to be pretreated, the filtration works being periodically subjected to a declogging operation; the operation of disinfecting the filtration works (F) is carried out during a declogging operation, and the liquid used for the declogging is formed of an aqueous declogging solution the salt content of which differs from the content of the water being pretreated; the bactericidal agent is added (14) to the declogging solution upstream of its injection into the filtration works (F).

The invention relates to a method for disinfecting a filtration works for the pretreatment of salt water, particularly seawater, upstream of a water desalination unit using a reverse osmosis membrane, the method being of the kind in which an operation of disinfecting the filtration works is carried out periodically by adding a bactericidal agent to the water that is to be pretreated, the filtration works being periodically subjected to a declogging operation.

The bactericidal agent generally consists of an oxidant, particularly chlorine, or chlorine dioxide.

It is an especial object of the invention to set in place a solution to the problem of disinfecting pretreatment works upstream of a reverse osmosis process, the purpose of this being to limit the biofouling (fouling with biological matter) of the reverse osmosis membranes, which is associated with these membranes becoming colonized with a biofilm.

The application of reverse osmosis processes on seawater entails the use of a pretreatment in order to protect the osmosis membranes from clogging. Clogging may be linked to the presence, in the feed water, of particles or of dissolved matter, either organic or inorganic. The clogging of the reverse osmosis membranes may also be linked with the development of a biofilm on the membrane itself or on the spacer, which is a constituent component of the reverse osmosis module that allows optimum hydraulic conditions to be maintained.

These pretreatments generally consist of a filtration step, using a granular medium or a membrane, which may or may not be associated with pre-coagulation of the particles and of the organic matter in order to encourage their separation.

Because seawater is a medium rich in micro-organisms, its passage through the pretreatment works, raw water feed pipes, filtration works, etc., may lead to the colonization of the surfaces with a biofilm. This biofilm is a source of seeding of the water, and leads to releases of organic matter which are byproducts of the metabolism of the micro-organisms that make up the biofilm and may, through the detachment of certain parts of this biofilm, once again increase the particles content of the pretreated water, with a risk of clogging the reverse osmosis membranes.

In order to limit this phenomenon of the colonization of the works, disinfection is generally performed, using a bactericide, generally an oxidant such as chlorine, chlorine dioxide or chloramines. In the vast majority of marine works, essentially for supplying electricity production facilities or desalination facilities using distillation processes, the water uptake works are protected from micro-organism colonization by the continuous addition of one of these oxidants. This continuous disinfection is often enhanced by shock chlorinations using a higher disinfectant residual periodically and randomly in order to kill off more highly evolved organisms such as molluscs.

The main disadvantage with this continuous chlorination technique is the formation of dissolved organic carbon which can be more readily assimilated by the micro-organisms from the organic carbon already present in the seawater or brine. Upstream of the reverse osmosis treatment, the bactericidal residual is generally neutralized to protect the membranes from contact with a powerful oxidizing agent: the micro-organisms may then develop on the surface of the membranes in the presence of biodegradable organic carbon, especially since the nutrient concentration is high. Continuous disinfection by using an oxidizing agent therefore acts as if to promote the development of the biofilm on the reverse osmosis membranes, and thus cause them to become clogged.

In order to confront this difficulty of performing effective disinfection of the pretreatment works and notably of the filters, according to the invention, a disinfection method of the kind defined hereinabove is characterized in that:
the operation of disinfecting the filtration works is carried out during a declogging operation,
and the liquid used for the declogging is formed of an aqueous declogging solution the salt content of which differs from the content of the water being pretreated, and the bactericidal agent is added to the declogging solution upstream of its injection into the filtration works.

The aqueous declogging solution may have a salt content higher than that of the water being treated; this aqueous declogging solution is advantageously formed of the discharge, or concentrate, from the reverse osmosis.

The declogging of the filter may also be performed using an aqueous solution the salt content of which is lower than the content of the water being pretreated.

The filtration for the pretreatment may be carried out in a granular materials filter particularly a dual layer filter; declogging then corresponds to an operation of washing the filtration works.

The filtration for the pretreatment may be performed in a unit with a membrane, in which case the declogging corresponds to a backwashing operation.

For preference, the bactericidal agent is chosen from among chlorine, chlorine dioxide and chloramines.

The method is thus based on a disinfection during washing or backwashing mode, combining the action of a disinfection with that of an osmotic shock created by the aqueous declogging solution that has a different salt content.

The invention also consists in a plant for implementing a method as defined hereinabove, which comprises a filtration works for a pretreatment of salt water, particularly seawater, and a water desalination unit with reverse osmosis membrane situated downstream of the filtration works, which plant comprises a set of pipes and valves for periodically carrying out an operation of declogging the filtration works, this plant being characterized in that:
it comprises a means for introducing, by way of washing liquid during the declogging operation, an aqueous solution the salt content of which differs from that of the water being pretreated,
and a means of injecting bactericidal agent into the washing liquid before it is injected into the filtration works.

For preference, the plant comprises a connecting pipe connecting the discharge side of the reverse osmosis unit to the filtration works, for injecting a washing solution that is more concentrated in salt than is the water being pretreated.

The filtration works for the pretreatment may consist of a granular materials filter, notably a dual layer filter, or a membrane unit. The membrane unit may have a submerged membrane or a membrane in a pressurized casing.

This technique has several advantages in the way in which it is coupled and implemented.

The use of an osmotic shock based on the discharge of the reverse osmosis process makes it possible to reduce the water losses of the plant by using a fluid that is continuously available at high flow rates in order to perform a washing action.

Coupling a bactericidal agent such as chlorine, DBNPA (dibromonitrilopropionamide) or chlorine dioxide with osmotic shock based on a hypersaline solution makes it possible surprisingly to increase the effectiveness of the cleaning: the inventors are of the belief that this increase in effectiveness is related to an increase in the effectiveness with which the bactericidal agents are disseminated within the biofilm. Specifically, the direct osmosis action combined with the introduction of a hypersaline solution associated with the bactericidal agent in a solution of lower salinity leads to accelerated dissemination of the bactericidal agent. Further, the joint action of the direct osmosis effect and of the bactericidal agent on the bacterial cells leads to a reduction in the CT (concentration C of bactericidal agent×T=contact time) needed for disinfecting the works.

Use of this novel device or system for disinfecting works is carried out during the step of periodic declogging of the filter, of washing the granular media filters with water or of backwashing membrane filtration systems. That makes it possible to avoid water that has been brought into contact with the bactericidal agent, and which therefore contains a higher concentration of biodegradable organic carbon, being sent into the reverse osmosis system. The time of contact between the hypersaline bactericidal solution associated with the bactericidal agent and the medium to be disinfected, granular medium of a sand or multi-layer filter, or a micro, ultra or hyperfiltration filtration membrane can be adjusted to suit the level of contamination of the system, simply by introducing a waiting time, a pause during the rinsing.

The injection of bactericidal agent may be halted before the end of rinsing. The system may be finally rinsed with filtered or ultrafiltered water in order to eliminate the hypersaline solution. This is chiefly the case for application to membrane filtration systems which do not require any maturation phase before re-entry into production for reverse osmosis. For systems employing granular materials, a maturation phase by drainage is generally carried out in order to eliminate the initial water that may contain an excess of particles, turbidity, residual oxidizing agent or biocide, excess salinity and the biofilm oxidation product.

Apart from the provisions set out hereinabove, the invention consists in a certain number of other provisions that will be dealt with more fully hereinafter with reference to some exemplary embodiments described with reference to the accompanying drawing, but which are not in any way limiting. In this drawing:

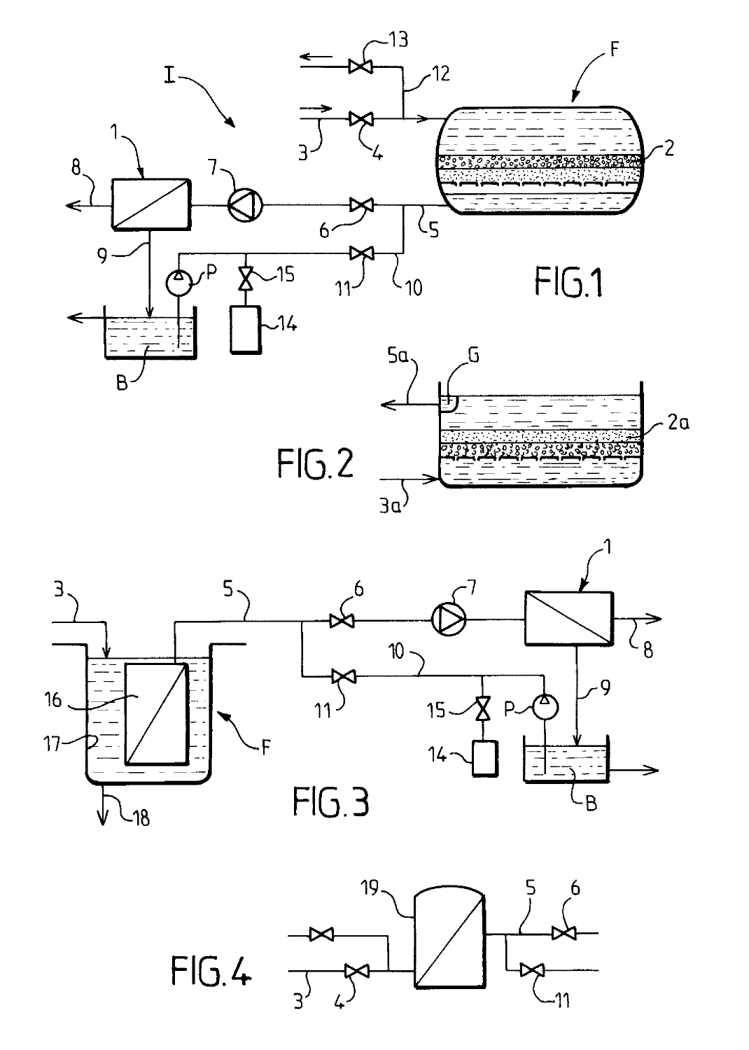

FIG. 1 is a diagram of a plant employing the method of the invention, with a closed granular materials filtration works.

FIG. 2 is a schematic cross section through an open filtration works constituting one possible alternative form of the filtration works of FIG. 1.

FIG. 3 is a simplified diagram of a plant employing the method of the invention with a filtration works consisting of submerged membranes, and

FIG. 4 shows an alternative form of the filtration unit of FIG. 3, with a membrane in a pressurized casing.

Reference is made to FIG. 1 of the drawing which shows a plant I for desalinating water, particularly seawater, and which comprises a water desalination unit 1 with reverse osmosis membrane and, upstream of this unit, a filtration works F for pretreating the salt water.

According to the example of FIG. 1, the filtration works F consists of a granular materials filter 2, notably a dual-layer filter, in a pressurized casing. The water for treatment arrives in the filter 2, in the upper part according to FIG. 1, via a pipe 3 equipped with a valve 4. The pretreated water leaves the filter 2, at the bottom, through a pipe 5 connected via a valve 6 to the intake side of a pump 7. The pretreated water is sent under pressure by the pump 7 to the reverse osmosis unit 1. The desalinated treated water leaves the unit 1 via a pipe 8. The discharge from the unit 1, consisting of water with a high salt concentration, also known as a hypersaline solution, leaves via a pipe 9 to be collected in a tank B forming a reserve, which is fitted with an overflow at the top.

For a periodic operation of declogging the filter 2, the plant comprises a pipe 10 for injecting a washing liquid into the filter 2. The pipe 10 is connected to the outlet side of a pump P via a valve 11. The intake side of the pump P is connected to the tank B for pumping the discharge. The washing liquid is removed by a pipe 12 connected to the pipe 3 between the valve 4 and the filter 2. This pipe 12 is fitted with a valve 13.

The plant further comprises a means 14 of injecting a bactericidal agent into the washing liquid before it is injected into the filter 2. The means 14 may consist of a pump or of a cylinder and piston metering device. The injection means 14 is connected, via a valve 15, to that part of the pipe 10 that lies between the valve 11 and the pump P.

The way in which the plant I works is as follows.

During normal periods of reverse osmosis desalination, the valves 4 and 6 are open while the valves 11, 13 and 15 are closed. Water for treatment arrives through the pipe 3 and enters the filter 2 where it undergoes pretreatment. The pretreated water leaves via the pipe 5 and is sent, under pressure, by the pump 7, into the reverse osmosis unit 1. The desalinated treated water leaves via the pipe 8, while the discharge leaves via the pipe 9.

When it is considered necessary to disinfect the filter 2, the disinfection operation is carried out during the declogging of the filter 2. The pump 7 is switched off, while the pump P is switched on. The washing liquid used for declogging is then formed of the discharge from the reverse osmosis unit the salt content of which differs from (namely is higher than) the content in the water being pretreated. The bactericidal agent is added to the declogging solution by opening the valve 15 and switching on the injection means 14. The bactericidal agent advantageously consists of chlorine or chlorine dioxide or chloramines. The washing solution is removed by the pump pipe 12 the valve 13 of which is open.

As an alternative, the declogging of the filter 2 could be performed using an aqueous solution the salt content of which is lower than the content of the water being pretreated.

Combining a bactericidal agent such as chlorine or chlorine dioxide with the osmotic shock created by the washing liquid the salt concentration of which differs from the concentration of the water being pretreated, makes it possible surprisingly to increase the efficiency of the cleaning and of the disinfection: this increase in efficiency seems to be connected with an increase in the efficiency with which the bactericidal reagents are disseminated within the biofilm. Further, the joint action of the direct osmosis effect and of the bactericidal agent on the bacterial cells leads to a reduction in the CT needed to disinfect the works.

FIG. 2 illustrates an alternative form of open filter 2a with granular materials that could be used in place of the filter 2 in FIG. 1. Water for pretreating arrives in the bottom part of the filter 2a via the pipe 3a. The pretreated water leaves via a pipe 5a at the top, connected to a trough G for collecting the pretreated water.

FIG. 3 shows a plant in which the pretreatment filtration system takes the form of a unit with a membrane 16 that is submerged in a basin 17. The other elements of the plant which are similar to those already described with reference to FIG. 1 are denoted by the same numerical references and not described again.

The permeate from the unit 16 constitutes the prefiltered water collected by the pipe 5. A discharge 18 is provided at the bottom of the basin 17 to discharge the concentrate from the unit 16.

The operation of disinfecting the unit with membrane 16 is carried out during a backwashing of the membrane with the liquid discharged from the reverse osmosis unit 1, with bactericidal agent 14 being injected into this backwash liquid.

During this disinfection operation, the pump 7 is switched off, the valve 6 is closed, while the valves 11 and 15 are opened. The pump P is switched on to backwash the membrane 16 with discharge liquid into which the bactericidal agent is injected by the means 14.

FIG. 4 shows an alternative form of the membrane filtration system whereby the membrane unit, rather than being submerged in a basin, forms a system in a pressurized casing 19.

Exemplary applications of the invention will now be described.

### EXAMPLE 1

### Application to a Granular Medium Filter (FIG. 1)

Simultaneous disinfection was carried out on a prototype with a granular materials filter consisting of two layers, in accordance with French patent application 06/11376 filed on 26 Dec. 2006 in the name of the same applicant company. The medias used were a layer of anthracite on top of a layer of sand.

The filter was fed with seawater that had previously been coagulated by the addition of ferric chloride (3 mg/l of FeCl₃) and subjected to acidification by the addition of sulphuric acid in order to drop the pH to the optimum value for coagulation of this water, which in this case was 6.8.

The rate of filtration applied was 12 m³/m²/h over the surface of the media. The duration of the filtration cycles that achieve a 3.4 m water column clogging of the filter bed was 22.5 h. The periodic washing of the filter was performed in accordance with the method described in French patent application 06/11376. During the phase of rinsing with water alone, which was performed using a hypersaline solution consisting of the concentrate from a reverse osmosis system, a disinfectant, namely chlorine, was injected at a concentration of 10 mg/l for 10 minutes each day. The CT was therefore 10×10×7=700 min·mg/l per week.

The bactericidal concentration of the filter feed water, of the filtered water, and of the biofilm collected in the filter was monitored under various operating conditions:
A: periodic washing of the filter with filtered water
B: periodic washing of the filter with filtered water to which a bactericidal agent, in this case chlorine at a concentration of 10 mg/l of active chloride had been added
C: periodic washing of the filter with reverse osmosis concentrate at a concentration of 75 g/l total salinity
D: periodic washing of the filter with reverse osmosis concentrate at a concentration of 75 g/l total salinity to which a bactericidal agent, in this instance chlorine at a concentration of 10 mg/l of active chloride had been added.
E: periodic washing of the filter with reverse osmosis concentrate at a concentration of 75 g/l total salinity to which a bactericidal agent, in this instance chlorine at a concentration of 5 mg/l of active chloride had been added. In this case, the addition of bactericidal agent was restricted to 5 min out of the 10 min of total rinsing time.

Table 1 sets out the results obtained (results averaged over a test period of one week for each period) where CFU stands for colony-forming units.

TABLE 1
Total flora count.

| | | | Residual biofilm prior |
|---|---|---|---|
| | Feed water | Filtered water | to periodic washing |
| | CFU/ml | CFU/ml | CFU/g of biofilm |
| A | 10² | 10³ | 5 · 10⁶ |
| B | 2 · 10² | 50 | 6 · 10³ |
| C | 1.5 · 10² | 1.5 · 10² | 3.4 · 10⁴ |
| D | 2.1 · 10² | 5 | 2 · 10² |
| E | 2 · 10² | 5 | 5 · 10² |
| A′ | 5 · 10² | 8 · 10³ | 4 · 10⁶ |

It was found during case A that the filter media became contaminated by the development of a biofilm. The bacteriological quality of the filtered water was degraded in comparison with the water being filtered because of the release of free bacteria and of fractions of biofilm.

Cases B and C use the addition of a bactericidal agent in the filter washing solution or used a hypersaline solution with which to wash the filter, making it possible to reduce the release of bacteria into the filtered water and the micro-organisms concentration in the biofilm present at the surface of the filtration medium.

Case D, implementing the invention, demonstrates a very marked improvement in the efficiency of disinfection through the joint use of washing with a hypersaline solution and the addition of a bactericidal agent.

Case A′, which reproduces the conditions of case A, confirms the colonization of the filter and the bacteriological degradation of the filtered water in a condition of washing without hypersaline solution and without bactericidal agent.

Case E, the results of which correspond to a CT which is halved in comparison with case D, once again has significant bactericidal activity in comparison with cases B and C.

### EXAMPLE 2

### Case of Application to a Membrane Filter (FIG. 3)

The disinfection method was applied to the operation of a membrane filtration system. The system used was a system with a submerged membrane, but could just as well be applied to a system in a pressurized casing. The membrane solution operated in direct seawater filtration mode without the addition of reagents. The filtration flow applied during the test was 50 l/m²/h.

Backwashing was performed every 30 minutes by counter-permeation of water in the opposite direction to the direction of filtration for 40 seconds. When 5 mg/l of chlorine were injected, the CT was therefore:

5×(40/60)×2×24×7=1120 min·mg/l

Four tests were carried out in the following configurations:
W: periodic backwashing of the filter with ultrafiltered water
X: periodic backwashing of the filter with ultrafiltered water to which a bactericidal agent, in this instance chlorine at a concentration of 5 mg/l of active chlorine had been added
Y: periodic backwashing of the filter with reverse osmosis concentrate at a concentration of 75 g/l total salinity
Z: periodic backwashing of the filter with the reverse osmosis concentrate at a concentration of 75 g/l total salinity to which a bactericidal agent, in this instance chlorine at a concentration of 5 mg/l of active chlorine had been added.

TABLE 2
Total flora count

| | Feed water | Ultrafiltered water |
|---|---|---|
| | CFU/ml | CFU/ml |
| W | 10⁴ | 100 |
| X | 2 · 10⁴ | 5 |
| Y | 1.5 · 10⁴ | 50 |
| Z | 2.1 · 10⁴ | <1 |
| W′ | 5 · 10⁴ | 155 |

In the case of ultrafiltration, the membrane catches in excess of 6 log of bacteria, the membrane cutoff threshold, in this instance 0.03 microns, being very much smaller than the diameter of the bacterial cells. Contamination on the permeate side occurred over the course of time when backwashing was performed with permeate alone (case W). This contamination is linked with the intrusion of bacteria during backwashing, which bacteria develop on the permeate side of the UF membranes.

The addition of a bactericidal agent or the use of a hypersaline solution with which to perform the backwashing of the membranes makes it possible to limit this contamination (cases X and Y).

The combining of the two solutions: hypersaline water with bactericidal agent, for the same contact time, yields a very marked reduction in the contamination of the permeate (case Z).

Case W′ corresponds to case W and demonstrates the recontamination of the permeate side of the membrane after a time of operation with neither hypersaline solution nor bactericidal agent.

1. Method for disinfecting a filtration works for the pretreatment of salt water, particularly seawater, upstream of a water desalination unit using a reverse osmosis membrane, whereby an operation of disinfecting the filtration works is carried out periodically by adding a bactericidal agent to the water that is to be pretreated, the filtration works being periodically subjected to a declogging operation, wherein:
the operation of disinfecting the filtration works is carried out during a declogging operation,
and the liquid used for the declogging is formed of an aqueous declogging solution the salt content of which differs from the content of the water being pretreated, and the bactericidal agent is added to the declogging solution upstream of its injection into the filtration works.

2. Method according to claim 1, wherein the aqueous declogging solution has a salt content higher than that of the water being treated.

3. Method according to claim 2, wherein the aqueous declogging solution is formed by the discharge (9, B) from the reverse osmosis.

4. Method according to claim 1, wherein the declogging of the filter is performed using an aqueous solution the salt content of which is lower than the content of the water being pretreated.

5. Method according to claim 1, in which the filtration for the pretreatment is carried out in a granular materials filter, wherein the declogging corresponds to an operation of washing the filtration works.

6. Method according to claim 1, in which the filtration for the pretreatment is carried out in a unit with a membrane, wherein the declogging corresponds to a backwashing operation.

7. Method according to claim 1, wherein the bactericidal agent is chosen from among chlorine, chlorine dioxide, DBNPA or chloramines.

8. Plant for implementing a method according to claim 1, comprising a filtration works for a pretreatment of salt water, particularly seawater, a water desalination unit with reverse osmosis membrane situated downstream of the filtration works, and a set of pipes and valves for periodically carrying out an operation of declogging the filtration works, wherein:
it comprises a connecting pipe connecting the discharge side of the reverse osmosis unit to the filtration works, and a pump, for injecting a washing liquid that is more concentrated in salt than is the water being pretreated,
and a means of injecting bactericidal agent into the washing liquid before it is injected into the filtration works.

9. Plant according to claim 8, wherein the filtration works for the pretreatment consists of a granular materials filter, particularly a dual layer filter.

10. Plant according to claim 8, wherein the filtration works for the pretreatment consists of a unit with a submerged membrane, or with a membrane in a pressurized casing.
